(19) United States
(12) Patent Application Publication
(10) Pub. No.: US 2003/0225345 A1
(43) Pub. Date: Dec. 4, 2003

(54) System and method for improving skin texture monitored with a proof device

(75) Inventors: Craig Stephen Slavtcheff, Guilford, CT (US); Christine Elizabeth Kennedy, Torrington, CT (US); Paul James Baker, Milford, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/370,855

(22) Filed: Feb. 20, 2003

Related U.S. Application Data
(60) Provisional application No. 60/385,493, filed on Jun. 4, 2002.

(51) Int. Cl. A61B 10/00

(52) U.S. Cl. 600/572

(57) ABSTRACT

A cosmetic product system is provided which includes a cosmetic composition for improving skin texture, and a test device packaged with the composition. The device includes a mechanism for visualizing progressive improvement in texture imparted by the composition as the latter is applied over a period of time.

### BACKGROUND OF THE INVENTION

1. Field of the Invention

The invention concerns a cosmetic composition for smoothing texture of skin and a test device packaged with the composition to demonstrate proof of its efficacy.

2. The Related Art

A number of publications have disclosed test devices for the lay person to self-diagnose their skin conditions. U.S. Pat. No. 3,571,947 (Maddison et al.) discloses a system for identifying blemishes. A flexible, compliant film of plastic is imprinted with pictorials of various types of common blemishes. These reflect different dermal diseases. They are cross-referenced with a handbook identifying the diseases from the type of blemish. Cross-indexed treatments are suggested to remedy the medical condition.

U.S. Pat. No. 5,727,949 (Bar-Or et al.) provides a dual ring panel reference card. The panels are mounted for relative movement whereby a selected diagnostic characteristic of a skin problem can be aligned with a second diagnostic characteristic and a determinable prognosis revealed from the specific paired characteristics.

CuDerm Corporation has developed a simple diagnostic test to determine the degree of skin dryness. CuDerm utilizes adhesive discs (D-Squame®) capable of removing a small section of squameous cells (skin cells) and compares the results against a chart. The disc is a transparent plastic with adhesive on one side. The test involves placing the adhesive surface of the disc against a user's forehead, peeling off the disc and placing same on a dark background card. Flakes from the skin stick to the adhesive surface and are visualized against the dark background. The greater the number of loose flakes, the dryer is the skin.

D-Squame® adhesive disks have been used in conjunction with Oil of Olay® and Dove® products to demonstrate before and after product use improvement against dry skin.

Considerable technology has also been developed with respect to measuring sebum concentrations. CuDerm Corporation markets a product branded Sebutape® for visualizing oiliness. U.S. Pat. Nos. 4,532,937, 5,088,502 and 5,119,828, all to Miller describe the Sebutape® product. It is structured as a microporous, hydrophobic polymeric film formed with pores and opaque to light. The pores are filled with gaseous material. When sebum fills those pores, the film turns from opaque to substantially translucent. The film is mounted to a substrate which includes a light absorbing area for enhancing visualization of the film pores when filled with sebum. Other sebum test devices are disclosed in U.S. Pat. No. 5,433,214 (Brehm et al.), U.S. Pat. No. 4,981,145 (Goldstein), U.S. Pat. No. 5,094,248 (Kawam) and U.S. Pat. No. 3,906,933 (Tur et al.).

There are many cosmetic products sold which advertise certain skin benefits. Consumers usually cannot easily discern whether the claimed benefit is actually delivered. Even if perceivable, these actives impart an effect which may emerge only slowly over a period of time. Improving skin to obtain a smooth texture is particularly illustrative. Certain types of skin benefit agents such as alpha-hydroxyacids and ceramides may be effective providing some visible improvement over an extended application period. They don't function instantaneously.

Accordingly, it is an advantage of the present invention to provide a cosmetic product system and method whereby progress in imparting a smoother texture to skin with a cosmetic product is measured by a low cost simple test for a consumer to self evaluate efficacy of the product.

Another advantage of the present invention is to provide a cosmetic product system and method employing a low cost simple self evaluation tool for measuring changes in skin smoothness and texture on the face or other parts of the human dermis.

### SUMMARY OF THE INVENTION

A cosmetic product system is provided which includes:

(i) a cosmetic composition effective to texturize skin, the composition exhibiting a Luminance Value of at least about 5%; and

(ii) a test device packaged with the composition, the device having means for visualizing progressive improvement in texture imparted by the composition, the means including at least three visualization units for time spaced-apart placement over an area of skin being monitored.

Also provided is a method for proof of efficacy of a cosmetic composition directed at improving skin texture which includes:

(a) providing a test device packaged with the composition, the device having means for visualizing progressive improvement in texture imparted by the composition, the means including at least three visualization units;

(b) providing a cosmetic composition effective to texturize skin when applied over a period of days, the composition exhibiting a Luminance Value of at least about 5%;

(c) placing one of the units on an application area of the skin and removing for assessing texture;

(d) applying the cosmetic composition to the application area;

(e) placing a further one of the units over the application area and removing for a second assessment of texture;

(f) re-applying the cosmetic composition to the application area; and

(g) placing yet another one of the units over the application area and removing for a third assessment of texture.

Among possible test devices, a most preferred embodiment is that of a proof tape. The tape can include a strip based on a water-insoluble substrate and an adhesive layer deposited onto the substrate. The adhesive layer will have sufficient tack to lift flecks of dry skin cells from a surface of the human body. Preferably the adhesive layer will be insufficient to develop any imprint of fine lines or wrinkles. In the first embodiment, the substrate will be transparent. Once flecks have been severed from the skin surface, the substrate is imaged by placing same against a darkened background. Advantageously there can be a series of 3, 4, 5 or more darkened areas on a proof card, each area being designated in print by a time or day on which an image was taken. An alternative embodiment may utilize a dark opaque substrate with adhesive. The resultant flecked substrate is a self imaged system. Preservation of the image may be accomplished by placement of a transparent sheet over the flecked adhesive layer. In this embodiment, the transparent non-adhesive cover sheet serves as a fixative.

One aspect of the present invention requires a consumer to save the image for a period of time as a comparison against a subsequent test image. Testing may occur initially and thereafter at regular intervals such as at 3, 7, 10 and 14 days or more. The time intervals and numbers may be longer or shorter. Therefore, it is desirable to fix the taken image to preserve same at least for a period of several days if not weeks. One example has already been illustrated above.

In the alternative embodiment of a dark opaque adhesive coated substrate, fixation can occur by providing the substrate with a chemical interactive with the fleck imaged opaque adhesive layer. Hardening can then occur between the chemicals of the dark background and those of the adhesive. For instance, the reactions may be oxidation-reduction, acid-base or polymerization in nature.

Alternative fixation can occur with adhesives that are UV or even natural/fluorescent light sensitive. A light penetration preventive protective peel-off strip covers the light sensitive adhesive, the latter supported on a substrate sheet. In operation, the light protective sheet is removed, the adhesive surface applied to the treatment area of the face, an image is formed through interaction between the dry skin flecks and the adhesive and then the resultant image is exposed to light. Exposure cures polymers forming the adhesive into a hardened material. Normally the supporting substrate bears a darkened color to contrast the image.

Another aspect of the present invention provides a system wherein a cosmetic composition is packaged with a test device. A variety of packaging arrangements are envisioned. The test device may be in the form of a cellulosic, plastic or combined material strip or tape placed into a carton alongside a container holding the cosmetic composition. Alternatively the test device may be incorporated as a panel segment of a carton, the latter protectively surrounding the cosmetic composition. In a variation thereof, the test device may be detachably joined to the package through a perforated or weakened construction line, or through an adhesive joinder.

### BRIEF DESCRIPTION OF THE DRAWING

Additional objects, features and benefits of the present invention will become more readily apparent from consideration of the drawing in which:

FIG. 1 is a first embodiment of a test device according to the present invention;

FIG. 2 is a second embodiment of a test device according to the present invention;

FIG. 3 is a third embodiment of a test device according to the present invention;

FIG. 4 is a fourth embodiment of a test device according to the present invention; and

FIG. 5 is the device of the embodiment shown in FIG. 1 subsequent to being placed on the skin, removed therefrom and mounted on a darkened field reading card.

### DETAILED DESCRIPTION OF THE INVENTION

Now there is provided to consumers a system for cosmetically transforming rough skin into a smooth evenly textured surface, a key indicator of healthy skin. The system includes a cosmetic composition packaged together with a simple diagnostic test device. Effectiveness of the product can be monitored by the consumer over a period of time through which the composition is applied on a regular basis.

In a preferred embodiment, the test device employs a transparent plastic strip coated with a transparent adhesive layer. When applied to a target area of the face or body, the adhesive layer accepts a topological imprint. Removal of the strip from this target area can then be imaged by placement onto a dark and, preferably black field.

Advantageously, a fixative is useful for maintaining a developed image of a roughened surface for an extended period of time. Fixation can be chemical in nature. For instance, adhesives can be blended with UV or natural light or fluorescent sensitive activatable monomers or oligomers. Light is shielded from the adhesive by an opaque strip covering the curable adhesive surface. Once the adhesive has contacted the skin and formed a texture pattern, the pattern is exposed to UV or natural or fluorescent light to harden the impression.

Another test device system employs an adhesive deposited onto a blackened substrate. The latter is contacted against the target skin. Upon removal, the adhesive surface with its image is overlain with a transparent sheet. The latter fixes the image against destruction.

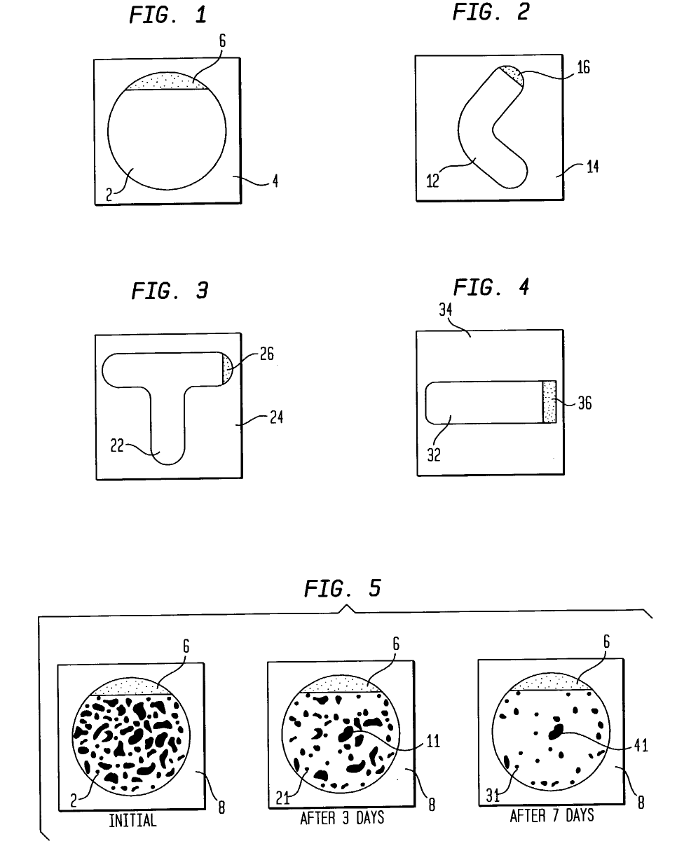

FIG. 1 illustrates a transparent strip 2 adhesively attached to a release backing 4. A tab 6 is attached to the strip 2. The tab serves as a gripping structure. Separation of the strip from the release backing is facilitated by initiating removal at the tab. The opaque, preferably black or white coloration of the tab in contrast to the transparency of the strip signals to a user the difference of this area and cues the user to start lifting at that point.

FIG. 2 illustrates a second shape embodiment. A strip 12 is formed as an L-shape. The strip includes a release backing 14 and a tab 16. This shape is particularly useful around the eye, ear and hands areas.

FIG. 3 illustrates a third embodiment. Here a strip 22 has a T-shape. The strip is adhesively attached to a release backing 24 and includes a tab 26 for easy grippable removal. The T-shape is particularly suitable around areas of the forehead and nose.

FIG. 4 illustrates a fourth embodiment. The strip 32 is adhesively attached to a release backing 34. Tab 36 operates as a gripping structure. The strip is a rectangular elongate shape with filleted edges. This shape is particularly useful for fingers and along areas of the neck. Comparably shaped dark imaging areas on a reading card or the like may be provided for this embodiment.

In the procedure for testing efficacy of various cosmetic products, the strip is removed from its release backing. Thereupon it is placed along an area of the skin to be imaged for evaluation of texture. Facial areas are primarily intended for evaluation, and more particularly areas around the cheek. Subsequently, the strip is removed and placed upon an imaging card 8. The dark, preferably black background of the card fixes the imprint while the transparent strip allows a view of that imprint.

FIG. 5 illustrates the strip showing a before use, a 3-day after use and a 7-day after use imprint. These require three separate units of the proof tape or test strip device. There is a gradual lessening of aggregated fleck areas and not just a reduction of overall fleck numbers as the cosmetic product progressively improves texture over the 3 to 7 day period.

Subsequent to a baseline analysis of texture, treatment is begun with a selected cosmetic texture improving composition. Treatment is continued for a period of time sufficient to allow the composition to improve skin smoothness. A second imaging field is placed adjacent to the first. After the first treatment, such as about 3 days later, another imprint is taken by a second transparent strip 21. If the cosmetic product is properly functioning, not only fewer flecks but most importantly fewer clumped aggregated flecks 11 with a more even distribution will appear on the imaged second field. This procedure can then be repeated at a further interval after more of the cosmetic composition has treated the skin surface. Each test will employ a fresh strip and a new blackened area on the same or another image card. The third transparent strip 31 is expected to show even fewer aggregated clumped flecks 41 than the earlier imprint.

With the particular illustrated embodiment, the adhesive preferably is insufficiently mobile to flow into skin crevices. This would prevent imaging of fine lines and wrinkles which might interfere with analysis of the surface dry cell image.

Strips for use with the illustrated preferred embodiment will be transparent articles allowing observation of any patterns on a surface thereof. Suitable materials for the strip are plastics or cellulosics of any variety which can be formed as transparent films. Typically the plastic may be selected from polyethylene, polypropylene, polystyrene, polyester, polycarbonate, polyacrylate, polyvinyl chloride, polyvinyl alcohol and polybutene. Not only homopolymers but copolymers may be utilized for the strip material. Copolymers may be formed from such monomers as C₂-C₁₀ olefins, vinyl chloride, acrylates and styrene constructed through free-radical polymerization. Condensation plastics may also be utilized in the formation of copolymers wherein the monomers may be selected from C₂-C₁₀ dicarboxylic acids, C₂-C₁₀ polyols, C₂-C₆ alkoxylates and combinations thereof. Polyethylene, polypropylene and polyester terephthalate are the preferred plastic substrates for forming the strip.

The thickness of the strip may range anywhere from about 0.00001 to about 2 mm, preferably from about 0.0001 to about 1 mm, more preferably from about 0.001 to about 0.5 mm and optimally from about 0.01 to about 0.1 mm.

The backing is typically made from a material and in a manner that is generally impervious to the adhesive. The backing may be elastic or non-elastic but preferably the former. Flexibility allows easier removal of the adhesive strip. The backing can be formed from a variety of materials including organic polymers and cellulosics. A release coating such as a silicone may be placed on an upper surface of the backing to ease removal of the adjacent adhesive strip.

The adhesive may be a pressure sensitive or non-pressure sensitive type preferably as a layer with an average thickness from about 0.000005 mm to about 2 mm, preferably from about 0.00005 mm to about 0.5 mm, more preferably from about 0.0005 mm to about 0.25 mm, optimally from about 0.005 mm to about 0.05 mm.

Pressure sensitive adhesives suitable for use in this invention are coatable adhesives. A wide variety of coatable pressure sensitive adhesives can be used, such as solvent coatable, hot melt coatable, as well as latex PSA's that are coatable out of water. Also, solventless curable adhesives (often referred to as 100% solids) can be used. Where thicker adhesive coatings are desired, it may be desirable either to apply multiple layers of the adhesive, hot melt coat, or to photopolymerize the adhesive in situ. Specific examples of pressure sensitive adhesives include acrylates, such as isooctyl acrylate/acrylic acid copolymers, tackified acrylates, and plasticizer-containing acrylates such as those disclosed in U.S. Pat. No. 4,946,742 (Landin); natural or synthetic rubber resins, including thermoset rubbers as well as thermoplastic rubbers and elastomers, such as nitrile rubbers (e.g., acrylonitrile-butadiene), styrene-butadiene, styrene-isoprene, styrene-butad iene-styrene, styrene-i soprene-styrene, and natural rubber; silicone-based adhesives, such as polysiloxanes; polyolefins; polyesters; polyamides; and polyurethanes.

Particularly preferred are the acrylic type pressure sensitive adhesives. Most especially a pressure sensitive adhesive with a low tack value.

Non-pressure sensitive adhesives are illustrated by polysaccharides. Examples are starches, chemically modified starches and natural or synthetic gums. Starches include corn and potato starches. Chemically modified starches include hydroxyalkylated starch, acylated starch, hydroxypropyl cellulose, hydroxypropyl methyl cellulose, ethyl cellulose and carboxymethyl cellulose. Gums include alginate, guar, carrageenan, agar, Karaya, pectin, gum arabic, sclerotium, gellatin and gum combinations.

The preferred strip embodiment according to the present invention is commercially available from the CuDerm Corporation under the trademark D-Squame®.

Cosmetic compositions of the present invention may be in the form of creams, lotions, pastes, sticks (e.g. lipsticks), or powders. These cosmetics normally will include a carrier. Suitable carriers include water, emollients (esters, hydrocarbons, silicones, polyols and mixtures thereof, emulsifiers, thickeners and combinations thereof. Most often the carrier will be an emulsion such as an oil-in-water or water-in-oil type. Amounts of the carrier may range from about 1 to about 99.9% by weight.

Cosmetic compositions of the present invention will be effective to texturize skin. These compositions will exhibit a Luminance Value of at least about 5%, preferably from about 5 to about 50%, optimally from about 10 to about 30%. The term “Luminance Value” is the mean change in percentage of grey area on a D-Squame® imaged by digital camera difference between a baseline value of skin and three days after treatment with the cosmetic composition.

Texture improving actives may include retinoids, ceramides, alpha or beta-hydroxycarboxylic acids, flavonoids, vitamins, anti-oxidants and mixtures thereof.

Typical retinoids include retinol, retinoic acid and retinol esters. The latter include retinyl palmitate, retinyl linoleate, retinyl propionate, retinyl acetate and retinyl salicylate.

Alpha-hydroxy acids include the free acid, lactone and salt forms of glycolic acid, lactic acid, citric acid, gluconolactone, glucarolactone, tartaric acid, malic acid and mixtures thereof. Beta-hydroxycarboxylic acids are exemplified by salicylic acid as well as its esters (e.g. tridecylsalicylate) and salts including ammonium, alkanolammonium and alkalimetal salts.

Ceramides include Ceramide 1, Ceramide 2, Ceramide 3, Ceramide 3a, Ceramide 3b, Ceramide 4, Ceramide 5 and Ceramide 6, as well as pseudoceramides, phytosphingosines and tetraacetyl phytosphingosine.

Vitamins may include ascorbic acid as well as its water-soluble and water-insoluble derivatives. Illustrative are ascorbyl tetraisopalmitate, magnesium ascorbyl phosphate and ascorbyl glucoside. Other vitamins include Vitamin B3 (niacin, niacinamide and panthenol), biotin, folic acid, tocopherol and its esters (e.g. tocopherol isopalmitate), Vitamin D and combinations thereof.

Antioxidants include BHT (butylated hydroxytoluene), BHA (butylated hydroxyanisole), hydroquinone, ferulic acid and esters thereof, green tea extract, lipoic acid, N-acetyl cysteine, resveratrol and combinations thereof.

Amounts of the texture improving actives may range anywhere from 0.0000001 to 30%, preferably from 0.0001 to 15%, more preferably from 0.1 to 5%, optimally from 0.5 to 2% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

All parts, percentages and proportions referred to herein and in the appended claims are by weight unless otherwise illustrated.

### EXAMPLE

This Example illustrates the method for evaluation of Luminance Value. The following composition was found to have a Luminance Value suitable for use in a system according to the present invention.

| INGREDIENT | WEIGHT % |
|---|---|
| Phase A | |
| Water | Balance |
| Disodium EDTA | 0.10 |
| DI-Panthenol | 0.20 |
| Sodium Lactate | 0.10 |
| Sodium PCA (50% Active) | 0.20 |
| Glycerin | 5.00 |
| Phase B | |
| Carbopol Ultrez 10 ® | 0.50 |
| Phase C | |
| Triethanolamine (99%) | 2.00 |
| Water | 10.00 |
| Parsol HS ® | 2.00 |
| Phase D | |
| Myristyl Alcohol | 0.50 |
| Sorbitan Stearate | 1.10 |
| Cetyl Alcohol | 0.50 |
| Glycery Dilaurate | 0.50 |
| Stearyl Alcohol 0.50 | |
| Sucrose Stearate | 0.25 |
| PEG-100 Stearate | 0.50 |
| Stearic Acid | 0.25 |
| Parsol MCX ® | 5.50 |
| Parsol 1789 ® | 2.00 |
| Dermablock OS ® | 3.00 |
| Vitamin E Acetate | 0.20 |
| Sunflower Seed Oil | 0.10 |
| Linoleic Acid | 0.10 |
| Cholesterol | 0.20 |
| Phase E | |
| Isododecane | 4.50 |
| Polymethacrylate Beads | 0.50 |
| Phase F | |
| Water | 0.50 |
| Ceramide/Phytosphingosine | 0.01 |
| Herbal Extract | 0.10 |
| Phase G | |
| lodopropynyl Butyl Carbamate | 0.10 |
| Nipaguard BPX ® | 0.20 |
| Merguard 1200 ® | 0.125 |
| Phase H | |
| Bisabolol | 0.20 |
| Vitamin A Palmitate | 0.01 |
| Fragrance | 0.30 |

A group of 50 panelists were each provided with a tape guard consisting of three D-Squame® test disks mounted to a black background. The panelists applied one of their three D-Squame® disks to skin at the back of their hand before product use (“0” baseline), after 3 days (one use of product per day), and after 7 days (one use of product per day).

Each sample was evaluated for exfoliation using an image analysis technique. The “0” use results were compared against the “3” use results, then the “3” use results were compared against the “7” use results. This comparison was done for each of the 50 panelists.

D-Squame® disks were imaged using a Kodak DCS-420 digital camera in monochrome mode. The camera was positioned above the sample using a photographic copy stand. The camera was mounted with a 105 mm-macro lens and a polarizing filter.

An illuminating lamp was switched on and allowed to stabilize for 10 minutes. Specular reflections were reduced by cross-polarizing the illumination with a polarizing filter at the light source and a second polarizing filter rotated 90 degrees at the camera lens. Fiber optic light diodes were positioned and locked to provide even illumination across the sample from an angle of 30-45 degrees.

Camera focus and aperture were selected and locked for the sample series. A sample was positioned in the camera field of view. Fine focus was achieved by moving the entire camera body up and down the copy stand track. Cross-polarization was checked on the first image captured and histogram statistics were measured using Optimas program. The statistical data was extracted from the image, transferred and saved to a disk. The sample was then removed and procedure repeated upon each of the samples in a series.

When imaged in monochrome, the skin flakes adhering to D-Squame® tapes appear as grey particles against the black background of the mounting card. In cases where more skin particles adhere to the tape, image analysis recorded a higher Luminance Mean Grey Value across the field of view as opposed to cases where fewer skin particles adhered to the tape yielding lower Luminance Mean Grey Values. Results of the image analysis are reported in the Table below.

| | Luminance Mean Grey Value | | | Luminance Value | |
|---|---|---|---|---|---|
| | | After 3 | After 7 | % Change | % Change |
| Panelist | Before Use | Days | Days | Day 0-Day 3 | Day 3-Day 7 |
| Mean | 14.345 | 16.895 | 15.804 | 17.8% | −6.5% |
| (n = 50) | | | | | |
| Maximum | 52.827 | 51.650 | 57.309 | −2.2% | 11.00% |
| Minimum | 2.415 | 1.072 | 1.059 | −55.6% | −1.2% |
| Median | 12.069 | 11.579 | 15.014 | −4.1% | 29.7% |

Optimas 4.1, Contrast-127, Brightness-175
DCS 420 Digital Camera
105 mm@1:½ F4 1/15^{th} sec
600 × 600 ROI
Adobe Photoshop Levels Command, 31 Low 127 High

According to the Table, the mean luminance of all 50 panelist samples showed a grey value of 14.345 before product use. After 3 days the grey value increased to 16.895. At 7 days the grey decreased to 15.804. The mean skin particle density increase between baseline to day 3 was 17.8%. Thus, the Luminance Value was 17.8%. The tested composition was effective at improving texturization of skin.

The foregoing description and examples illustrate selected embodiments of the present invention. In light thereof variations and modifications will be suggested to one skilled in the art, all of which are within the spirit and purview of this invention.

What is claimed is:

1. A cosmetic product system comprising:
(i) a cosmetic composition effective to texturize skin, the composition exhibiting a Luminance Value of at least about 5%; and
(ii) a test device packaged with the composition, the device having means for visualizing progressive improvement in texture imparted by the composition, the means including at least three visualization units for time spaced-apart placement over an area of skin being monitored.

2. The system according to claim 1 wherein the test device comprises a water-insoluble substrate and a polymeric layer deposited onto the substrate.

3. The system according to claim 2 wherein the polymeric layer is an adhesive.

4. The system according to claim 3 wherein the polymeric layer is sufficiently adhesive to remove flecks of dry skin but of insufficient adhesivity to image wrinkles and thereby not impair visualization of the flecks.

5. The system according to claim 3 wherein the adhesive is a polymer selected from the group consisting of acrylates, starches, gums, polyvinyl alcohol and mixtures thereof.

6. The system according to claim 2 wherein the test device further comprises a protective cover substrate positioned over the polymeric layer, the cover substrate being removed prior to application of the polymeric layer against the area of skin being monitored.

7. The system according to claim 1 wherein the test device comprises a material selected from a cellulosic, plastic or combination material strip, and the strip is placed into a carton alongside a container holding the cosmetic composition.

8. The system according to claim 1 wherein the test device is incorporated as a panel segment of a carton protectively surrounding a container holding the cosmetic composition.

9. The system according to claim 1 wherein the test device is detachably joined to a carton protectively surrounding a container holding the cosmetic composition, joinder of the test device being through a means selected from group consisting of perforations, weakened carton wall and adhesive joinder.

10. A method for evaluating efficacy of a cosmetic composition directed at improving skin texture, the method comprising:
(a) providing a test device packaged with the composition, the device having means for visualizing progressive improvement in texture imparted by the composition, the means including at least three visualization units;
(b) providing a cosmetic composition effective to texturize skin when applied over a period of days, the composition exhibiting a Luminance Value of at least about 5%;
(c) placing one of the units on an application area of the skin and removing for assessing texture;
(d) applying the cosmetic composition to the application area;
(e) placing a further one of the units over the application area and removing for a second assessment of texture;
(f) re-applying the cosmetic composition to the application area; and
(g) placing yet another one of the units over the application area and removing for a third assessment of texture.
